(19) United States
(12) Patent Application Publication
(10) Pub. No.: US 2022/0133752 A1
(43) Pub. Date: May 5, 2022

(54) MINOCYCLINE FOR THE TREATMENT OF PITT-HOPKINS SYNDROME

(71) Applicant: HEALX LTD, Cambridge (GB)

(72) Inventor: David BROWN, Cambridge (GB)

(21) Appl. No.: 17/431,837

(22) PCT Filed: Feb. 26, 2020
(86) PCT No.: PCT/GB2020/050458

§ 371 Date: Aug. 18, 2021

(30) Foreign Application Priority Data
Feb. 26, 2019 (GB) 1902580.8

(51) Int. Cl. A61K 31/65; A61P 25/28

(52) U.S. Cl. CPC A61K 31/65; A61P 25/28

(57) ABSTRACT

The present invention relates to minocycline or a pharmaceutically acceptable sale thereof, for use in the treatment of Pitt-Hopkins syndrome.

### FIELD OF THE INVENTION

This invention relates the treatment of Pitt-Hopkins syndrome (PTHS).

### BACKGROUND OF THE INVENTION

Pitt-Hopkins syndrome is a rare, genetic neurological disorder caused by a molecular variant of TCF4 which is involved in embryologic neuronal differentiation.

PTHS is characterised by distinctive facial features (syndromic facies), developmental delay (psychomotor delay), intellectual disability, early-onset myopia, seizures, constipation and breathing abnormalities (hyperventilation-apneic spells i.e. recurrent episodes where they breathe very fast, often followed by episodes where they struggle to breathe or momentarily stop breathing) and low muscle tone (hypotonia). Further symptoms include repetitive nonfunctional hand movements and behavioural abnormalities such as hyperactivity and anxiety. Many affected individuals meet criteria for autism spectrum disorder.

PTHS is caused by a pathogenic variant of the TCF4 gene found on chromosome 18q21.2. The syndrome was first described in 1978 in two unrelated individuals who shared similar characteristics of dysmorphic facial features, developmental delay, clubbed fingers, and an abnormal breathing pattern. It was presumed to be an autosomal recessive disorder until 2007 when the TCF4 gene, (MIM #602272) was identified, supporting an autosomal dominant inheritance pattern secondary to haploinsuffiency of TCF4. Transcription factor 4, the protein product of TCF4, is a basic helix-loop-helix E-protein believed to be involved in early brain development and neuronal differentiation.

At present there is no effective therapy to treat PTHS. There is a need for therapies for the treatment of PTHS.

Minocycline is a tetracycline antibiotic used in the treatment of bacterial infections and acne vulgaris. Minocycline has the systematic name (4˜{S},4˜{a}˜{S},5˜{a}˜{R},12˜{a}˜{R})-4,7-bis(dimethylamino)-1,10,11,12˜{a}-tetrahydroxy-3,12-dioxo-4˜{a}, 5,5˜{a},6-tetrahydro-4˜{H}-tetracene-2-carboxamide.

### SUMMARY OF THE INVENTION

The present invention is minocycline, or a pharmaceutically acceptable salt thereof, for use in the treatment of PTHS. As will be evident from the in vivo data presented below, minocycline is effective in treating PTHS. Chronic treatment with minocycline significantly improved the Tcf4+/− mouse phenotype, rescuing fear conditioning, open field, nesting, self-grooming, sociability and test of force. This is evidence that minocycline is useful in the therapy of PTHS.

A first aspect of the invention is minocycline, or a pharmaceutically acceptable salt thereof, for use in the treatment of Pitt-Hopkins syndrome.

### DESCRIPTION OF THE FIGURES

FIG. 1 shows the results from minocycline in vivo testing for fear conditioning (learning and memory).

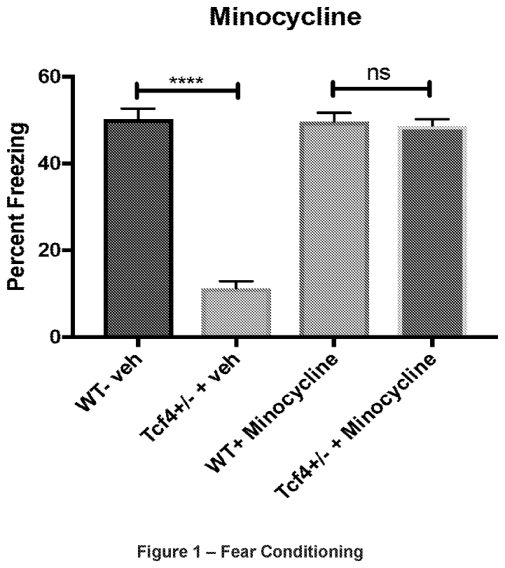

FIG. 2 shows the results from minocycline in vivo testing for open field (hyperactivity).

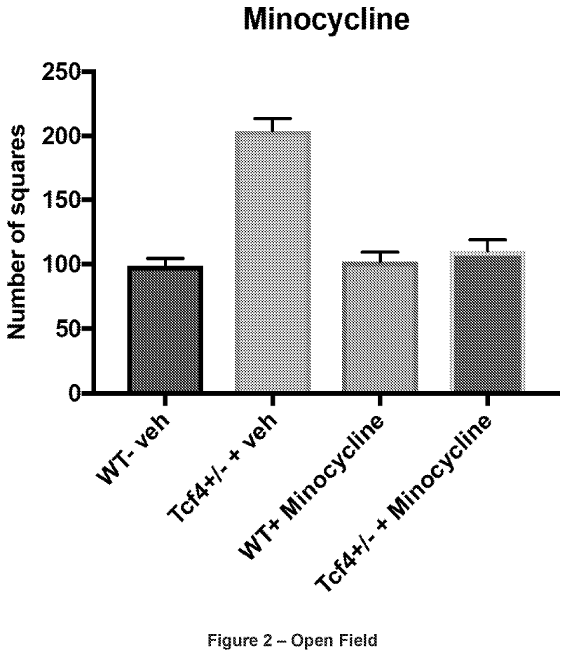

FIG. 3 shows the results from minocycline in vivo testing for nesting (tests of daily living).

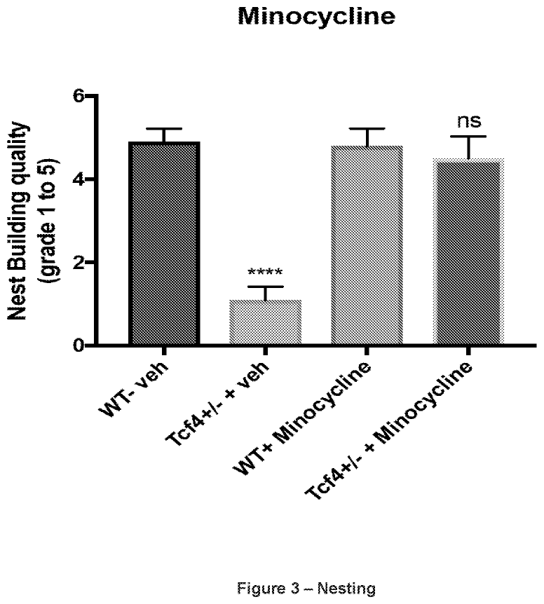

FIG. 4 shows the results from minocycline in vivo testing for elf-grooming (stereotypy i.e. repetitive movement).

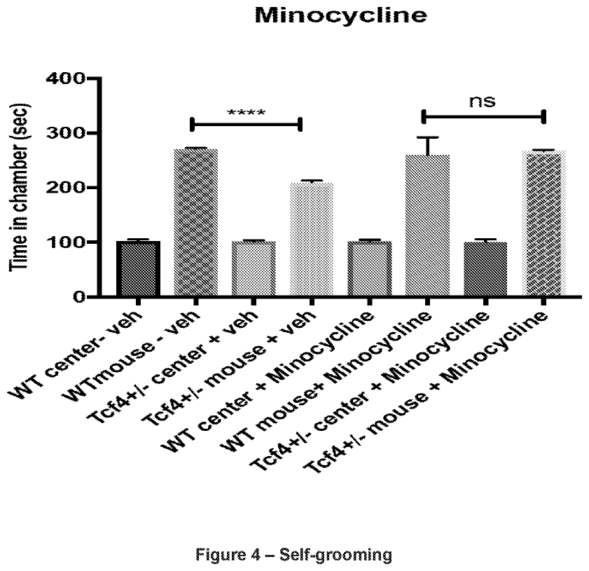

FIG. 5 shows the results from minocycline in vivo testing for sociability.

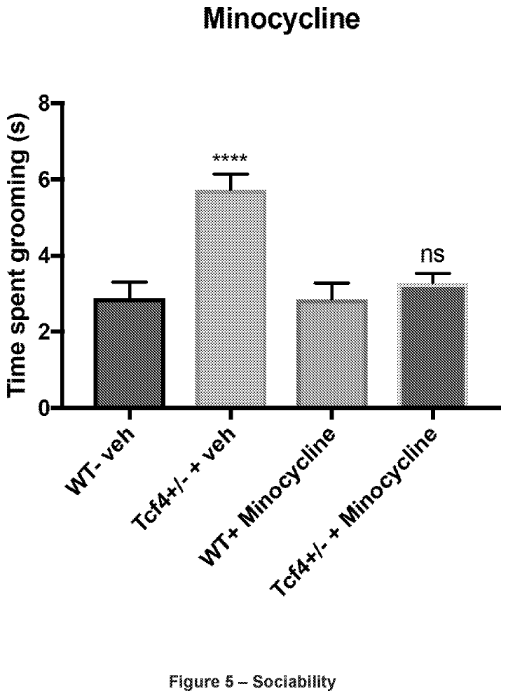

FIG. 6 shows the results from minocycline in vivo testing test of force (strength).

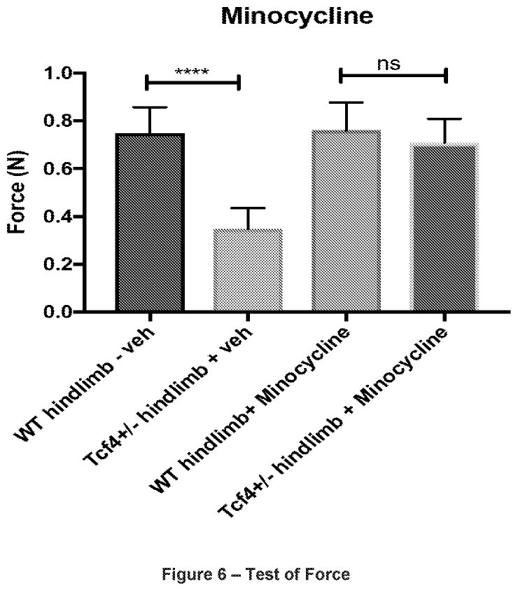

FIG. 7 shows the results of dose response experiments in 5 behavioural tests.

In the Figures, ‘ns’ means no significant difference from Wild Type control group. This indicates that the drug-treated Knock Out is behaving like the untreated or treated Wild Type group in the behavioural test, meaning the compound was significantly effective in ameliorating the syndrome's phenotype.

### DETAILED DESCRIPTION

There are a number of different manifestations and symptoms in patients with Pitt-Hopkins syndrome. These include: hyperactivity and repetitive behaviour including repetitive nonfunctional hand movements; intellectual impairment, such as difficulties with learning and memory e.g. difficulties with cognitive, executive and language performance, short-term memory, executive function, visual memory, visual-spatial relationships and spatial working memory; stereotypy; social anxiety (i,e, difficulties in social interaction); low muscle tone (hypotonia); constipation, sleep disturbances, seizures; irregular or abnormal breathing patterns and severe nearsightedness (myopia).

In the present invention, and as demonstrated by the below in vivo data, minocycline is used to treat one or more of the above symptoms, and is therefore an effective treatment of PTHS. Preferably, minocycline is used for the treatment of PTHS, wherein the patient is exhibiting typical symptoms of the syndrome including hyperactivity; social anxiety; intellectual impairment, specifically difficulties with learning and memory; stereotypy; and hypotonia.

The term “intellectual impairment” has its normal meaning in the art. It encompasses impairment in learning and memory. Learning impairment may also be called intellectual disability. It encompasses cognitive impairment, delay or limitations in intellectual functions, such as reasoning, Memory impairment refers to an inability to retain information either short-term or long-term. It may include difficulties with cognitive, executive and language performance, executive function and visual memory. It may also include difficulties with working memory, also called short-term memory (i.e. the temporary storage of information while processing the same or other information) and difficulties with phonological memory (or verbal working memory). This symptom was tested in mice under “fear conditioning” (see in vivo data below).

The term “hyperactivity” has its normal meaning in the art. Hyperactivity may include having very short attention spans_{;} hypersensitivity to visual, auditory, tactile, and olfactory stimuli, distractibility, impulsiveness, restlessness and/or over-activity. This symptom was tested in mice under “open field” (see in vivo data below).

The term “test of daily living” has its normal meaning in the art. It may also mean ability to perform the things normal to a species including any daily activity we perform such as bedding, feeding etc. This symptom was tested in mice under “nesting” (see in vivo data below).

The term “stereotypy” has its normal meaning in the art, It may also be termed as repetitive movements or repetitive behaviour. This symptom was tested in mice under “self-grooming” (see in vivo data below).

The term “social anxiety” has its normal meaning in the art. It may also be termed as difficulties in social interaction or low sociability. Social anxiety may include having poor eye contact, gaze aversion, prolonged time to commence social interaction, social avoidance or withdrawal and challenges forming peer relationships. This symptom was tested in mice under “sociability” (see in vivo data below).

The term “hypotonia” has its normal means in the art. This symptom was test in mice under “test of force”. The term “force” has its normal meaning in the art. It may also mean the strength or energy put into an action.

As used herein, a pharmaceutically acceptable salt is a salt with a pharmaceutically acceptable acid or base. Pharmaceutically acceptable acids include both inorganic acids such as hydrochloric, sulphuric, phosphoric, diphosphoric, hydrobromic or nitric acid and organic acids such as citric, fumaric, maleic, malic, ascorbic, succinic, tartaric, benzoic, acetic, methanesulfonic, ethanesulfonic, salicylic, stearic, benzenesulfonic or p-toluenesulfonic acid. Pharmaceutically acceptable bases include alkali metal (e.g. sodium or potassium) and alkali earth metal (e.g. calcium or magnesium) hydroxides and organic bases such as alkyl amines, aryl amines or heterocyclic amines.

In the present invention, minocycline may be administered in a variety of dosage forms. In one embodiment, minocycline may be formulated in a format suitable for oral, rectal, parenteral, intranasal or transdermal administration or administration by inhalation or by suppository,

Minocycline may be administered orally, for example as tablets, troches, lozenges, aqueous or oily suspensions, dispersible powders or granules. Preferably, minocycline is formulated such that it is suitable for oral administration, for example tablets and capsules.

Minocycline may also be administered parenterally, whether subcutaneously, intravenously, intramuscularly, intrasternally, transdermally or by infusion techniques. Minocycline may also be administered as suppositories.

Minocycline may also be administered by inhalation. An advantage of inhaled medications is their direct delivery to the area of rich blood supply in comparison to many medications taken by oral route. Thus, the absorption is very rapid as the alveoli have an enormous surface area and rich blood supply and first pass metabolism is bypassed.

The present invention also provides an inhalation device containing minocycline. Typically said device is a metered dose inhaler (MDI), which contains a pharmaceutically acceptable chemical propellant to push the medication out of the inhaler.

Minocycline may also be administered by intranasal administration. The nasal cavity's highly permeable tissue is very receptive to medication and absorbs it quickly and efficiently. Nasal drug delivery is less painful and invasive than injections, generating less anxiety among patients. By this method absorption is very rapid and first pass metabolism is usually bypassed, thus reducing inter-patient variability. Further, the present invention also provides an intranasal device containing minocycline.

Minocycline may also be administered by transdermal administration. For topical delivery, transdermal and transmucosal patches, creams, ointments, jellies, solutions or suspensions may be employed. The present invention therefore also provides a transdermal patch containing a minocycline,

Minocycline may also be administered by sublingual administration. The present invention therefore also provides a sub-lingual tablet comprising minocycline.

Minocycline may also be formulated with an agent which reduces degradation of the substance by processes other than the normal metabolism of the patient, such as anti-bacterial agents, or inhibitors of protease enzymes which might be the present in the patient or in commensural or parasite organisms living on or within the patient, and which are capable of degrading the compound.

Liquid dispersions for oral administration may be syrups, emulsions and suspensions.

Suspensions and emulsions may contain as carrier, for example a natural gum, agar, sodium alginate, pectin, methylcellulose, carboxymethylcellulose, or polyvinyl alcohol. The suspension or solutions for intramuscular injections may contain, together with the active compound, a pharmaceutically acceptable carrier, e.g. sterile water, olive oil, ethyl oleate, glycols, e.g, propylene glycol, and if desired, a suitable amount of lidocaine hydrochloride.

Solutions for injection or infusion may contain as carrier, for example, sterile water or preferably they may be in the form of sterile, aqueous, isotonic saline solutions.

In an embodiment of the invention, minocycline is administered in an effective amount to treat the symptoms of Pitt-Hopkins syndrome. An effective dose will be apparent to one skilled in the art, and is dependent on a number of factors including age, sex, weight, which the medical practitioner will be capable of determining.

In a preferred embodiment, minocycline is administered in doses of 1 mg to 300 mg, preferably 5 mg to 300 mg, more preferably 50 mg to 300 mg, most preferably 100 mg to 200 mg. The lower limit for a dose is preferably 1 mg, 2 mg, 3 mg, 4 mg or 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 130 mg, 140 mg, 150 mg, 160 mg, 170 mg, 180 mg, 190 mg or 200 mg. The upper limit for a dose is preferably 300 mg, 290 mg, 280 mg, 270 mg, 260 mg, 250 mg, 240 mg, 230 mg, 220 mg or 210 mg. Any of the aforementioned lower or upper limits of the ranges may be combined with each other, and are herein disclosed. Preferably the dose is 1 mg to 200 mg or 50 mg to 200 mg.

Any of the above doses may be administered once a day, twice a day, three times a day or four times a day.

In an embodiment of the invention, minocycline is administered at least once a day. Preferably it is administered as a single daily dose. Preferably the single daily dose is of 1 mg to 300 mg, preferably 100 mg to 300 mg. Preferably it is 100 mg, 200 mg, or 300 mg.

In an embodiment of the invention, minocycline is administered twice daily. Preferably each dose is 1 to 150 mg or 100 mg to 150 mg, with a total daily dosage of 300 mg,

Alternatively, it may be administered three times per day. Preferably each dose is 100 mg.

Alternatively, it may be administered four times per day. Preferably each dose is 75 mg.

Preferably, the dosage regime is such that the total daily dosage of minocycline does not exceed 300 mg.

It will be appreciated that a lower dose may be needed in a paediatric patient. For example, a dose of about 2 mg may be appropriate in a paediatric patient.

In order to treat Pitt-Hopkins syndrome, minocycline is used in a chronic dosage regime i.e. chronic, long-term treatment.

The present invention also relates to use of minocycline, or a pharmaceutically acceptable salt thereof, for the manufacture of a medicament for use in the treatment of Pitt-Hopkins syndrome. This embodiment of the invention may have any of the preferred features described above.

The present invention also relates to a method of treating Pitt-Hopkins syndrome comprising administering the patient with minocycline or a pharmaceutically acceptable salt thereof. This embodiment of the invention may have any of the preferred features described above. The method of administration may be according to any of the routes described above.

For the avoidance of doubt, the present invention also embraces prodrugs which react in vivo to give a compound of the present invention.

The following studies illustrate the invention.

### Study 1

Pitt-Hopkins syndrome (PTHS) is characterized by cognitive dysfunction, wide mouth and distinctive facial features, and intermittent hyperventilation followed by apnoea (Zweier et al., 2007). The cognitive dysfunction associated with the loss of one copy of the TCF4 gene, termed haplo-insufficiency, in humans leads to Pitt-Hopkins syndrome, an autism-related disorder associated with pronounced learning deficits.

Pitt-Hopkins syndrome (PTHS) is a genetic neurodevelopmental disorder associated with transcription factor TCF4 mutations/deletions. TCF4 may also be linked to schizophrenia, suggesting that the precise pathogenic mutations are relevant to cellular, synaptic, and behavioural consequences.

The Tcf4 +/− mouse model has been developed to mimic PTHS and therefore assess potential treatments. The mice have deficits in hippocampus-dependent learning and memory, spatial working memory, sociability, stereotypy, hyperactivity and daily living paradigms. The Tcf4 +/− mice also demonstrate hindlimb grip strength deficits.

### Animals

Mice were purchased from The Jackson Laboratory and maintained on a C57BL/6 background. They were raised on a 12:12 light:dark cycle with ad libitum access to food and water. Controls consisted of TCF4 +/+. All mice used were heterozygous for the Tcf4 mutation because homozygous mutations result in embryonic /post-partum lethality.

### Assay Design

Experiments were randomised and blind to the genotype and treatment during all testing and data analysis. Separate investigators prepare and coded dosing solutions, allocate the mice to the study treatment groups, dosed the animals, and collect the behavioural data.

### Treatment Groups

There were four treatment groups per compound in the study with 10 male mice used per treatment group (all at 14 weeks of age): Group 1: wild-type littermate mice treated with vehicle (WT+veh); Group 2: Tcf4 +/− KO mice treated with vehicle (Tcf4 +/− +veh), Group3: WT+drug; and Group 4: Tcf4 +/− +drug.

### Behavioural and Strength Tests

This included:

1. Fear conditioning (a test of learning and memory);

2. Open field (a measure of hyperactivity)

3. Nesting (a test of daily living)

4. Self-grooming (an assessment of stereotypy).

5. Sociability/Partition test (an assessment of social anxiety)

6. Test of Force (a test of hind limb strength).

The term “learning and memory” has its normal meaning in the art. It may also be called memory impairment. It refers to an inability to retain information either short-term or long-term. It may include difficulties with cognitive, executive and language performance, executive function and visual memory. It may also include difficulties with working memory, also called short-term memory (i.e. the temporary storage of information while processing the same or other information) and difficulties with phonological memory (or verbal working memory).

The term “hyperactivity” has its normal meaning in the art. Hyperactivity may include having very short attention spans, hypersensitivity to visual, auditory, tactile, and olfactory stimuli, distractibility, impulsiveness, restlessness and/or over-activity.

The term “test of daily living” has its normal meaning in the art. It may also mean ability to perform the things normal to a species including any daily activity we perform such as bedding, feeding etc.

The term ‘self-grooming’ has its normal meaning in the art. It may also mean self-cleaning and maintaining normal appearance of skin, hair, fur etc. Carried to excess via persistent repetition of an act it may be referred to as ‘sterotypy’.

The term “social anxiety” has its normal meaning in the art. It may also be termed as difficulties in social interaction or low sociability. Social anxiety may include having poor eye contact, gaze aversion, prolonged time to commence social interaction, social avoidance or withdrawal and challenges forming peer relationships.

The term “force” has its normal meaning in the art. It may also mean the strength or energy put into an action.

### 1. Fear Conditioning

Tcf4 +/− mice show reduced freezing in the context of clued fear conditioning test indicating hippocampus-dependent memory and learning deficiency. The dependent measure used in contextual fear conditioning was a freezing response following a pairing of an unconditioned stimulus (foot shock), with a conditioned stimulus, a particular context. Freezing is a species-specific response to fear, which has been defined as “absence of movement except for respiration”. This may last for seconds to minutes depending on the strength of the aversive stimulus, the number of presentations, and the degree of learning achieved by the subject. Testing involved placing the animal in a novel environment (dark chamber), providing an aversive stimulus (a 1-sec electric shock, 0.2 mA, to the paws), and then removing it.

### 2. Open Field

The open-field apparatus was used to test hyperactivity. Tcf4+/− mice show hyperactivity when compared to WT littermates in distance travelled per minute during a 30 minutes trial Open Field. The apparatus was a grey PVC-enclosed arena 50×9×30 cm divided into a 10×10 cm grid. Mice were brought to the experimental room 5-20 min before testing. A mouse was placed into a corner square facing the corner and observed for 3 min. The number of squares entered by the whole body (locomotor activity) were counted. The movement of the mouse around the field was recorded with a video tracking device for 3 min (version NT4.0, Viewpoint).

### 3. Nesting

The test was performed in individual cages. Normal bedding covered the floor to a depth of 0.5 cm. Each cage was supplied with a “Nestlet,” a 5 cm square of pressed cotton batting (Ancare). Mice were placed individually into the nesting cages 1 hr. before the dark phase, and the results were assessed the next morning. Nest building was scored on a 5 point scale.
Score 1: The Nestlet was largely untouched (>90% intact).
Score 2: The Nestlet was partially torn up (50-90% remaining intact).
Score 3: The Nestlet was mostly shredded but often there was no identifiable nest site: <50% of the Nestlet
Score 4: An identifiable, but flat nest <90% of the Nestlet was torn up, the material was gathered into a flat nest with walls higher than the mouse height curled up on its side) on less than 50% of its circumference.
Score 5: A (near) perfect nest: >90% of the Nestlet was torn up, the nest was a crater, with walls higher than mouse body height on more than 50% of its circumference.
4. Self-Grooming

Tcf4 +/_ mice groom themselves significantly more than WT mice indicating higher levels of stereotype behaviour than control mice.

Usually in a sitting position, the mouse will lick its fur, groom with the forepaws, or scratch with any limb. Often the mouse will mix all of these grooming behaviors. Grooming typically follows a sequence of four behaviors:
Elliptical Stroke: Elliptical asymmetric movements of the forepaws over the nose and muzzle, alternating the major and minor paw.
Unilateral Stroke: Alternating strokes of the forepaw across the vibrissae and the eye.
Bilateral Stroke: Large symmetric bilateral strokes of the forepaws that begin behind the ears and pass over the whole face.
Body Licking: Licking of the whole body, typically beginning rostrally and working caudally to the tail.

### 5. Partition Test

In the three-chambered sociability task, a subject mouse was evaluated for its exploration of a novel social stimulus (novel mouse). The three-chambered social approach task monitors direct social approach behaviours when a subject mouse is presented with the choice of spending time with either a novel mouse or an empty cup. Sociability is defined as the subject mouse spending more time in the chamber containing the mouse than in the empty chamber. Preference for social novelty is defined as spending more time in the chamber with the novel mouse. The apparatus is a rectangular three-chamber box, where each chamber measures 20 cm (length)×40.5 cm (width)×22 cm (height), Dividing walls are made from clear perplex, with small openings (10 cm width×5 cm height) that allow access into each chamber. The three chamber task was lit from below (10 lux). The mice were allowed to freely explore the three-chamber apparatus over three 10 min trials. During the trial one wire cup was placed upside down in one of the side chambers and a novel mouse was placed under another wire cup in the other side chamber (novel mouse stimulus), leaving the middle chamber empty. The location of the novel mouse across trials was counterbalanced to minimize any potential confound due to a preference for chamber location. The time spent exploring the novel mice was scored as exploration ratio.

### 6. Test of Force

Tcf4+/− mice show a significant deficit in hindlimb strength (not forelimb) when compared with VvT mice. Neuromuscular function of the hindlimbs was tested with a grip strength meter (San Diego Instruments). Mice were scruffed by the back of the neck, held by the tail, and lifted into an upright position. Then, the mice were lowered toward the apparatus, allowed to grasp the smooth metal grid (hindlimbs only), and pulled backwards in the horizontal plane. The force applied to the grid at the moment the grasp was released was recorded.

### Statistical Analysis of Behavioural Data

Data were analysed by two-way analysis of variance (ANOVA) followed by post-test comparisons where appropriate using Tukey's Multiple Comparison Test. Data are represented as the mean and standard error of the mean (SEM).

### Conclusion

As evident from the data above, chronic treatment of Tcf4+/− mice with minocycline significantly improved learning and memory; hyperactivity; tests of daily living; stereotypy; social anxiety and test of force. As the mouse model mimics PTHS, this is evidence that minocycline has a therapeutic effect for PTHS.

### Study 2

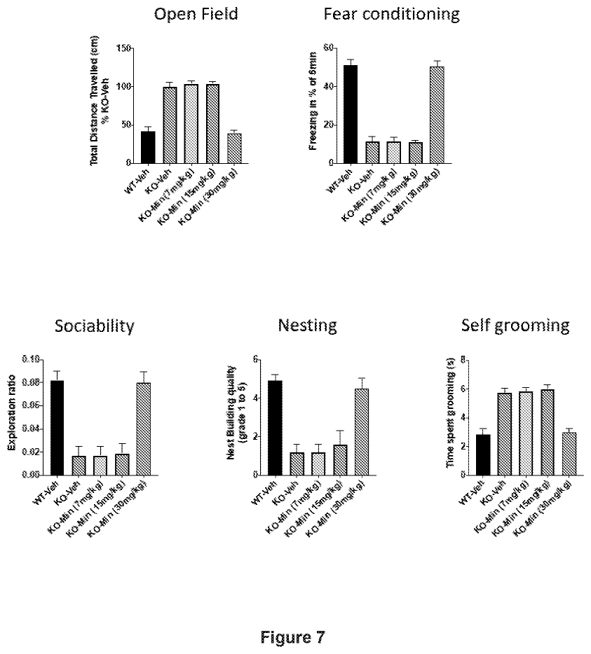

Dose response experiments were conducted following the protocol described above. The results are shown in FIG. 7. Minocycline rescued all the behavioral tests at 30 mg/kg.

1-12. (canceled)

13. A method of treating Pitt-Hopkins syndrome, comprising administering minocycline or a pharmaceutically acceptable salt thereof to a patient.

14. The method of claim 13, wherein the patient is human.

15. The method of claim 13, wherein the minocycline is administered in an amount selected from 50 mg to 300 mg and 100 mg to 200 mg.

16. The method of claim 13, wherein administration is by a single daily dose.

17. The method of claim 16, wherein the single daily dose is 200 mg to 300 mg.

18. The method of claim 13, wherein administration is by a dose twice per day.

19. The method of claim 18, wherein each dose is 100 mg to 150 mg.

20. The method of claim 13, wherein administration is oral.

21. The method of claim 13, wherein administration is by a route selected from parenteral, transdermal, sublingual, rectal and inhalation.

22. The method of claim 13, wherein the patient is exhibiting signs of intellectual impairment, hyperactivity, stereotypy, social anxiety and/or hypotonia.
